(19) United States
(12) Patent Application Publication
(10) Pub. No.: US 2010/0191346 A1
(43) Pub. Date: Jul. 29, 2010

(54) BONE IMPLANT

(76) Inventors: Stephen Bloor, Alwoodley (GB); Joanne Louise Proffitt, Alwoodley (GB); Paul Armitage, Ackworth (GB); Christine Elizabeth Dawson, Byram (GB)

Correspondence Address: Tyco Healthcare Group LP, 60 MIDDLETOWN AVENUE, NORTH HAVEN, CT, 06473, US

(21) Appl. No.: 12/596,177

(22) PCT Filed: Apr. 15, 2008
(86) PCT No.: PCT/GB08/01327

§ 371 Date: Mar. 24, 2010

(30) Foreign Application Priority Data
Apr. 16, 2007 (GB) 0707235.8
Oct. 31, 2007 (GB) 0721347.3

(51) Int. Cl. A61F 2/28; C12N 5/077

(52) U.S. Cl. 623/23.63; 435/381

(57) ABSTRACT

A bone implant derived from natural bone tissue material, wherein the bone implant is substantially free of non-fibrous tissue proteins, cells and cellular elements and lipids or lipid residues and comprises collagen displaying original collagen fibre architecture and molecular ultrastructure of the natural bone tissue material from which it is derived.

The present invention relates to a bone implant prepared from natural bone tissue.

Certain orthopaedic procedures require bone implants or grafts to provide a scaffold for new bone growth or to act as filler, for instance where bone defects have been removed or repaired.

Healing in a primary bone wound involves similar stages to healing in other wounds, with the initial formation of haematoma, followed by an inflammatory reaction and polymorphonuclear leukocyte infiltration. The clot is invaded by macrophages and chemotactic agents attract bone marrow stromal cells and stimulate angiogenesis. Marrow stromal cells contain a small number of mesenchymal stem cells, which have the ability to differentiate into a variety of cell types depending on the local environment and regulatory factors.

Mesenchymal stem cells are fibroblastic in appearance, and it is these cells that actually migrate to the wound site. If conditions are not optimal for bone or cartilage formation, the cells may differentiate along a default pathway and become fibroblasts. When this occurs, non-union results.

Bone formation ('ossification') can generally be classified into two types, intramembranous and endochondral.

Intramembranous ossification takes place when a group of mesenchymal cells differentiate directly into osteoblasts. These cells synthesise a woven bone matrix, while at the periphery mesenchymal cells continue to differentiate into oseoblasts. Blood vessels are incorporated into the woven bone trabeculae and will form the hemotopoietic bone marrow. Later, the newly formed woven bone will be remodelled and replaced by mature lamellar bone.

Endochondral ossification begins when a group of mesenchymal cells form a cartilaginous model of the bone to be formed. Mesenchymal cells undergo division and differentiate into prechondroblasts and then into chondroblasts. These cells then secrete the cartilaginous matrix. Like osteoblasts, the chondroblasts become progressively embedded within their own matrix, where they lie within lacunae. They are then referred to as chondrocytes. Unlike osteocytes, chondrocytes continue to proliferate for some time and this is partly due to the gel-like consistency of cartilage. At the periphery of this cartilage, the mesenchymal cells continue to proliferate and differentiate.

Bone tissue can be laid down as either woven or lamellar bone. In rapidly-formed woven bone, the collagen fibrils that are manufactured by osteoblasts are distributed within the matrix in a random arrangement making woven bone mechanically weak. Woven bone is the first bone matrix formed in endochondral and intramembranous bone formation during skeletal growth and development, and is sometimes referred to as immature bone. It is usually only found in the adult skeleton in cases of trauma or disease, most frequently occurring around bone fracture sites.

Lamellar bone is bone in which the collagen fibrils are formed in extracellular spaces by osteoblasts and have an ordered arrangement. This is a mechanically stronger matrix compared to woven bone and is the type of bone found in the mature skeleton. Within the cortex, the lamellar bone is functionally arranged as virtually solid tubes centred upon a capillary in the cortex. These tubular structures are termed haversion systems or osteons.

When the ends of the bone are in close proximity and the bone is mechanically stable, osteochondroprogenitor cells are able to migrate across the haematoma and form bone directly. Following proliferation, these cells differentiate into osteoblasts, which synthesise and then calcify osteoid (uncalcified organic bone matrix, mainly made up of collagen) via a mechanism that involves matrix vesicles. This rapidly forming bone is termed woven bone because it lacks structural organisation.

The woven bone is eventually remodelled and replaced by lamellar bone. This process takes varying lengths of time depending on the site and whether the bone is in a mechanically active area. Generally, bone healing and remodelling requires at least six months, although this may be longer in complicated or large wounds.

Bone in human and other mammals can generally be classified into two types: cortical bone (sometimes referred to as compact bone) and cancellous bone (also known as trabecular or spongy bone). These two types of bone can be classified on the basis of porosity and the unit microstructure. Cortical bone is much denser with a porosity generally ranging between 5% and 30%. It is found primarily in the shaft of long bones and forms the outer shell around cancellous bone at the end of joints and the vertebrae. Cancellous bone is much more porous, with porosity ranging anywhere from 50% to 90%. It is found in the end of long bones, in vertebrae and in flat bones like the pelvis.

In instances where a large wound exists, a bone graft may be required to fill the wound and promote healing. Most often, autograft materials are used as they tend to be osteogenic, osteoconductive and non-immunogenic.

However, there are drawbacks and limitations of the use of autograft bone. For example, there is the additional surgical time required to harvest autograft bone, which increases operative risk. There is also additional injury to the patient caused by the bone harvesting procedure. This in turn can lead to a longer recovery time, due to the morbidity of the donor site, and increased postoperative pain. In addition, the amount of available bone suitable for harvesting is limited and there may not be a sufficient quantity to fill large defects. Furthermore, in certain circumstances it may not be possible to harvest any bone from the patient, or only a small quantity of bone may be obtainable. The orthopaedic surgeon will then require an alternative form of bone implant, either to ‘bulk out’ the available autograft material or to use in isolation as the graft.

An ideal bone graft substitute material should be osteoinductive, osteoconductive, resorbable, biologically compatible and have a proven safety profile with no adverse local or systemic effects.

Osteoconduction is the physical property of the graft to serve as a scaffold for viable bone healing. Osteoconduction allows for the ingrowth of neovasculature and the infiltration of osteogenic precursor cells into the graft site.

Osteoinduction is the ability of a material to induce stem cells to differentiate into mature bone cells. This process is typically associated with the presence of bone growth factors within the graft material or as a supplement to the bone graft.

Bone is a specialised connective tissue composed of both mineral and organic phases designed for its role as a load bearing structure of the body. To accomplish this task bone is formed from a combination of dense cortical bone and cancellous bone that reinforces areas of stress. Two principle cells are found in bone, the osteoclast and the osteoblast and both these cells are essential to the turnover and remodelling of bone. The osteoblast produces the matrix which becomes mineralised in a regulated manner, while the osteoclast is able to remove the mineralised matrix when activated. Bone is constantly undergoing remodelling which is a complex process involving the resorption of bone on a particular surface, followed by a period of bone formation. In normal adults there is a balance between the amount of bone resorbed by the osteclasts and the amount of bone formed by the osteoblasts. In addition to the normal remodelling of bone, both osteoclasts and osteoblasts are essential in bone healing.

Fracture healing restores the tissue to its original physical and mechanical properties and is influenced by a variety of systemic and local factors. Healing occurs in three distinct but overlapping stages: the early inflammatory stage; the repair stage; and the late remodelling stage.

In the inflammatory stage, a hematoma develops within the fracture site during the first few hours and days. Inflammatory cells (macrophages, monocytes, lymphocytes, and polymorphonuclear cells) and fibroblasts infiltrate the bone. This results in the formation of granulation tissue, ingrowth of vascular tissue, and migration of mesenchymal cells. The primary nutrient and oxygen supply of this early process is provided by the exposed cancellous bone and muscle.

During the repair stage, fibroblasts begin to lay down a stroma that helps support vascular ingrowth. Undifferentiated mesenchymal stem cells undergo rapid chondrogenesis, which is modified by endochondral ossification. As vascular ingrowth progresses, a collagen matrix is laid down while osteoid is secreted and subsequently mineralised, which leads to the formation of a soft callus around the repair site. In terms of resistance to movement, this callus is very weak in the first 4 to 6 weeks of the healing process and requires adequate protection in the form of bracing or internal fixation. Furthermore, early in the repair phase, new bone formation also occurs adjacent to old bone. This appositional bone growth resembles intramembranous ossification and forms a bridge spanning and surrounding the fracture site and the central cartilaginous callus. Chondrocytes within the callus cartilage mature by the same process as in endochondral bone growth, but in a more disorganised manner. Vascularisation of the callus, and the invasion of osteoclasts in the mineralised cartilage, also reflects the processes observed in endochondral bone growth. Osteoclasts degrade cartilage matrix until only thin spicules remain. Osteoblasts migrate to line the cavities formed and produce new woven bone matrix. Eventually, the callus ossifies, forming a bridge of woven bone between the fracture fragments. Fracture healing is completed during the remodelling stage in which the healing bone is restored to its original shape, structure, and mechanical strength.

Remodelling of the bone occurs slowly over months to years and is facilitated by mechanical stress placed on the bone. As the fracture site is exposed to an axial loading force, bone is generally laid down where it is needed and resorbed from where it is not. Adequate strength is typically achieved in 3 to 6 months.

Controlled remodelling of a bone substitute is important to its success at providing a strong and successful repair. Ideally, a bone substitute material should be remodelled as new bone is formed. If a bone substitute material remains in the defect site after bone healing is complete then it has the potential to alter the material properties of the bone, and its mechanical resistance to stress.

There are currently a number of bone graft products suitable for use in surgical procedures. The existing products include synthetic materials, processed bovine bone materials, and treated allograft materials, as outlined below.

Demineralised bone matrix (DBM) is a product of processed allograft bone. DBM is the best known and widely used example of an osteoinductive graft. DBM contains collagen, proteins and growth factors that are extracted from the allograft bone. It is available in the form of a powder, crushed granules, putty, chips or as a gel that can be injected through a syringe. DBM is extensively processed and therefore has little risk for disease transmission. However, because of the form it takes it does not provide strength to the surgical site.

DBM is prepared by decalcifying allograft bone to expose the organic matrix, along with a number of stimulatory chemical signalling factors trapped in the organic matrix during bone formation. The factors contained within the DBM are capable of causing mesenchymal stem cell chemotaxis, proliferation and differentiation, giving rise to new bone formation. Also, the underlying matrix provides a suitable scaffold for cell attachment.

The majority of DBM use is in the form of particulates (powders or fibres) requiring the use of a carrier to impart desirable handling properties to the graft. A variety of inert carriers have been used including glycerol and gelatine. These carriers are largely considered non-contributory to the biological events and work solely to improve the handling characteristics of the material.

A series of low molecular weight glycoproteins that include bone morphogenetic proteins (BMPs) are generally considered to be the most important bone growth factors contained in DBM, although other factors such as osteopontin, osteocalcin and osteonectin may also be important. The BMPs are considered to provide DBM with osteoinductive potential.

Although allograft bone materials have essentially all the same components as DBM (with the exception of the mineral content), they are not osteoinductive. Demineralisation of the allograft bone is required to impart this property. If allograft bone is implanted into a heterotopic site it is resorbed. If, however, it is implanted orthotopically, it is generally very effective.

Ceramics are highly crystalline structures formed by heating non-metallic mineral salts to high temperatures (≧1000° C.) in a process known as sintering. Calcium phosphate-based ceramic bone fillers are synthetic materials that have been used in dentistry since the 1970s and in orthopaedics since the 1980s. Ceramics offer no significant possibility for disease transmission, although they may be associated with inflammation in some patients. They are available in many forms, including porous and mesh forms. Although ceramics may provide a framework for bone growth, they contain none of the natural proteins that influence bone growth.

Hydroxyapatite (HA) is one of the families of calcium orthophosphate molecules, and is one of the most biologically compatible substances used as a bone graft substitute material. Although synthetic HA materials share similarities with the mineral phase of bone, they are very different. Bone mineral is highly carbonated and exists as very small plate-like crystals, in a three-dimensional matrix in dynamic arrangement with proteins and other extracellular matrix constituents. Synthetic HA is highly crystalline in structure and tends to be resorbed over a very long period of time.

Tricalcium phosphate (TCP) ceramic has a chemical reactivity similar to that of amorphous precursors to bone, whereas HA has a chemical reactivity which is closer to that of bone mineral. Neither of these synthetic mineral types occurs naturally. However, both are considered to be highly biocompatible and to evoke a biological response similar to that of natural bone, and both are known to be osteoconductive.

When these synthetic materials are immobilised next to healthy bone, osteoid is secreted directly onto the surfaces of the ceramic. Subsequently, the osteoid mineralises and the resulting new bone undergoes remodelling.

Differences do exists in the biological response by the host site to these different materials. In the case of porous TCP ceramic, the implant is removed from the implant site as new bone grows into the scaffold, whereas HA tends to provide a more permanent implant. Subtle differences in the chemical composition and crystalline structure of calcium phosphates may also have a major impact on the physical characteristics in vivo. Constructs with a higher density and crystallisation will have greater mechanical strength but undergo slower reabsorption.

The mechanical properties of calcium phosphate scaffolds are not suited to withstand the associated torsional and tensile forces imposed on the skeleton, and as such their use is limited to non-load bearing implantation sites. However, post-implantation their strength will increase as the porous structure of the material is penetrated by host tissue, eventually leading to the implant's mechanical strength reaching that of cancellous bone.

The porosity of the structure is a major determinate of the amount of surface area exposed to the biological environment. Greater porosity can accelerate the physical processes such as dissolution as well as biological processes, such as cell attachment and osteoid deposition. Therefore, the porosity of the implant is the primary physical determinate of the speed and completeness of incorporation of bone-forming tissue and subsequent bone remodelling.

Pore size is also an important characteristic of HA bone graft substitutes. Studies have shown that no in-growth occurs with a small pore size and fibrous tissue forms with a pore size of around 15 to 40 μm, whereas osteoids form with pore sizes of 100 μm. Pore sizes in the region of 150-500 μm are optimal for interface activity, bone growth and implant resorption.

The most commonly used bone grafts made from TCP are approximately 35% to 50% porous, with pores ranging from 100-300 μm. However, pore size may be less critical than the presence of interconnecting pores.

Interconnected porosity, found only in some calcium-based scaffolds, allows viable cellular components to permeate throughout the matrix to allow rigid fixation in the surrounding bone. These interconnecting pores also prevent the formation of ‘blind alleys’ at the bottom of which is found low oxygen tension, which prevents osteoprogenitor cells from following the osteoblast lineage cascade, differentiating instead into cartilage, fibrous tissue or fat.

Most HA-based grafts are osteoconductive, but when large blocks are used, even if highly porous, the ability of osteoprogenitor cells to migrate throughout the material may be compromised, and fibrous connective tissue may result. To overcome these problems, HA and other calcium phosphates may be used as composites with a more resorbable material, such as collagen or a synthetic biodegradable polymer.

Hydroxyapatite may also be made from natural coral exoskeletons, which are composed from calcium carbonate. Since these HA materials are not coral but are derived from the mineral content of coral, they are generally referred to as coralline.

Although there are hundreds of genera of stony corals, Porites and Goniopora are the only two that meet the required standards of pore diameter and interconnectivity. The exoskeleton of Porites is similar to that of cortical bone whereas the genus Goniopora is closer in structure to that of cancellous bone.

Two processes for manufacturing coralline materials exist. One approach is to use coral directly in the calcium carbonate form. These materials are called natural corals. The manufacturing process involves detergent aided cleaning to remove the organics and then sterilising the material with irradiation. The second process is known as replamineform, and converts the calcium carbonate to calcium hydroxyapatite.

Bone implants made from coral have been shown to be useful in the treatment of bone defects due to trauma, tumours and cysts. Such implants may also be used for spinal surgery as either a graft additive, or extender, or as an implant to provide a framework for bone to grow into.

Similar to the concept of the use of coral-derived material as bone graft substitutes, bovine bone can be processed to remove the organic components, leaving the structural properties of the mineral intact. The resulting pore size and porosity of the deproteinised bone is biologically compatible with normal bone. The deporoteinisation process involves heating the bovine bone material to remove the organic components within the structure.

Deproteinised bone has been developed as an alternative to autograft or allograft material using a variety of processing methods. At lower temperatures, many of the physical characteristics of the bone mineral are retained, whereas at higher temperatures, the mineral becomes sintered HA.

Studies have shown that bone processed at lower temperatures retains some organic material trapped within the mineral phase, including minute levels of biologically active osteogenic factors, which may contribute to the apparent clinical success of these bone graft substitutes.

However, the main attractive feature of bone processed at both low and high temperatures is the osteoconductive three-dimensional bone like morphology of the mineral material.

Composite graft materials have recently been developed in which combinations of bone grafting materials and/or bone growth factors are used to gain the benefits of a variety of substances. Among the combinations in use are a collagen/ceramic composite, which closely reproduces the composition of natural bone, DBM combined with bone marrow cells, which aid in the growth of new bone, and a collagen/ceramic/autograft composite.

BMPs are produced to regulate bone formation and healing. BMPs can speed up healing as well as limit the negative reaction to donor bone and the non-bone substitutes. BMPs guide modulation and differentiation of mesenchymal cells into bone and bone marrow cells.

The seminal paper reporting the initial discovery of BMP activity was published by Urist in 1965 (Science 1965, 150:893-899). Since then, the osteoinductive capacity of DBM has been well established. Acid demineralisation of allograft bone leaves behind a composite of non-collagenous proteins, collagen and most importantly osteoinductive bone growth factors.

BMPs make up only 0.1% by weight of all the bone proteins. Unlike DBM, which is a mixture of BMPs and noninductive proteins, the pure form of BMPs is non-immunogenic and non-species specific. BMPs have a number of functions ranging from extra cellular and skeletal organogenesis to bone regeneration. They cause mesenchymal cells to differentiate into chondrocytes, which create a cartilage matrix that mineralises and then is replaced by bone (endochondral ossification). Currently, single BMPs are available through recombinant gene technology, and mixtures of BMPs are available as purified bone extracts for clinical studies.

The present invention provides a new form of bone implant derived from natural bone tissue.

According to a first aspect of the present invention there is provided a bone implant derived from natural bone tissue material, wherein the bone implant is substantially free of non-fibrous tissue proteins, cells and cellular elements and lipids or lipid residues and comprises collagen displaying original collagen fibre architecture and molecular ultrastructure of the natural bone tissue material from which it is derived.

The bone implant is useful in the surgical treatment of a range of bone defects, including traumatic injuries or surgically created defects. The bone implant is typically substantially non-immunogenic and substantially non-cytotoxic.

Bone collagen predominantly comprises type I collagen molecules, which are assembled into collagen fibrils. Typically, these fibrils have a diameter of between 50 nm and 500 nm and are several micrometers in length. The collagen fibrils form bundles that in turn make collagen fibres. It is these fibres that provide structure to the bone tissue and provide additional mechanical properties to the inorganic mineral structure of the tissue.

It is particularly preferred that the bone implant retains at least part of the inorganic, mineral component of the natural bone tissue from which it is derived. The mineral component of the natural bone tissue is most preferably generally intact in the bone implant, i.e. the bone implant may be substantially non-demineralised (or in other words, substantially mineralised). By way of example, the bone implant as described herein may comprise approximately 10 to 95% organic material, being essentially collagen, typically approximately 20 to 75%, more typically approximately 22 to 50%, and still more typically approximately 25 to 35% organic material. The remainder of the bone implant comprises the inorganic material, being essentially hydroxyapatite. The inorganic material may typically include calcium phosphate, calcium carbonate, calcium fluoride, calcium hydroxide and citrate.

During natural bone development, the mineral element of the bone tissue is laid down upon a ‘scaffold’ formed by the organic matrix made up predominantly of type I collagen. By retaining the natural collagen structure along with at least part of the mineral component, the bone implant of the present invention is provided with good structural performance when compared to synthetic hydroxyapatite materials which can be relatively brittle due to the lack of a polymeric sub-structure to support the minerals.

Preferably, the mineral component of the bone implant retains generally its natural structure, i.e. the structure observed in the natural bone tissue material from which the bone implant is derived. Different bones differ in the structure of their inorganic matrices and therefore by selecting different starting materials it is possible to obtain bone implants with varying mineral component structures.

In certain particularly preferred embodiments, at least a portion of the bone implant comprises mineral wherein the structure of the collagen-mineral composite of the starting material is at least partially maintained. The natural bone tissue material, or a part thereof, may be processed so as to preserve as much as possible of the structure of the collagen-mineral composite forming the bone. Non-fibrous tissue proteins, cells and cellular elements and lipids or lipid residues are substantially removed from the natural bone tissue material to leave a composite of essentially collagen (with minor amounts of other fibrous tissue proteins) and mineral, in approximately the same arrangement as in the original natural bone tissue material. The collagen displays original fibre architecture and molecular ultrastructure seen in the collagen matrix present in the natural bone tissue material. The mineral component maintains architecture and relationship to collagen seen in the starting material.

Thus, a particularly preferred bone implant is derived from natural bone tissue material and is substantially free of non-fibrous tissue proteins, cells and cellular elements and lipids or lipid residues, comprises a collagen component displaying original collagen fibre architecture and molecular ultrastructure of the natural bone tissue material from which it is derived and further comprises a bone mineral component displaying original mineral architecture of the natural bone tissue material. The collagen component and bone mineral component of the bone implant preferably have a structural relationship approximating to the natural bone tissue material.

The preferred bone implant structure is an open network of connected bone trabeculae with a range of pore sizes and pore interconnectivity. Whereas cortical bone porosity tends to be quite low, for example around 5 to 30%, the porosity of trabecular bone varies, for example between around 50 to 90%. For instance, a previous study demonstrated that the porosity of trabecular bone from human mandibular condyles is around 79.3% (Renders, G. A., L. Mulder, L. J. van Ruijven, and T. M. van Eijden. 2007 Porosity of human mandibular condyler bone. J. Anat. 210:239-248). The porosity of the processed bone implant as described herein may vary accordingly, for example between around 5 to 90%, depending upon the starting materials. For any scaffold designed to augment bone replacement, certain characteristics are desirable, including an interconnected porous structure with a range of porosities to facilitate in growth, capillary infiltration, diffusion of nutrients and oxygen and removal of waste products. It has been shown in prior art studies that a pore size range from 100 μm to approximately 900 μm is suitable for tissue engineered bone (Salgado et al., 2004, Macromol. Biosci. 4:743-765). Some studies suggest a larger pore size (1.2-2 mm) is beneficial (Holy et al., 2000, J. Biomed. Mater. Res. 51:376-382), but a larger pore size may compromise the mechanical properties of the graft. The bone implant as described herein may comprise pores of any size, such as, for example, 1 μm to 2000 μm. By way of example, representative samples of the bone implant as described herein have been shown to have pores ranging from around 100 μm to around 1000 μm, which allow cellular infiltration without reducing the mechanical integrity of the structure.

Furthermore, to maintain the mechanical structure of bone it is preferable to preserve the original bone matrix architecture as far as possible. Since the preferred processing methods described herein do not greatly compromise the native architecture of the bone, the mechanical properties of the bone implant are comparable to that of human bone. In contrast, a prior art implant Orthoss® (Geistlich), although harvested from a cancellous source, is apparently altered by the processing techniques used during manufacture. In the Orthoss® implant, the organic components of the tissue including the collagen are removed from the bone structure through a process of chemical and high temperature treatments. The removal of the collagen affects the mechanical performance of the implant.

Advantageously, following implantation of the bone implant described herein, host bone tissue is laid down on the implant as lamellar bone, giving a good quality, strong repair. This is indicative of the implant being recognised by host cells as ‘natural’. The host bone tissue is formed on the implant mainly through intramembranous ossification as opposed to endochondral ossification. The new bone tissue is laid down directly onto the bone implant. Furthermore, the bone implant may be subject to resorption through the action of osteoclasts. Osteoclasts are large multinucleated cells that are responsible for the resorption of the bone matrix. They resorb natural bone by producing a mixture of hydrogen ions and hydrolytic enzymes such as Cathespin K. These dissolve and digest both the inorganic and organic aspects of bone. Therefore, compared to synthetic bone implants such as ceramics and synthetic hydroxyapatites, the bone implant exhibits a biological response closer to that of natural bone.

Surprisingly, the bone implant as described herein has been found to be not only osteoconductive but also osteoinductive. In other words, the bone implant not only acts as a passive ‘scaffold’ for the laying down of new bone tissue following implantation but also actively induces new bone formation in the host.

According to a further aspect of the present invention, there is provided a substantially non-demineralised bone implant derived from natural bone tissue material, wherein the bone implant is osteoconductive and osteoinductive.

It has been generally accepted that a non-demineralised (or mineralised) bone implant derived from natural bone tissue material does not provide an osteoinductive effect, being unable to induce stem cells to differentiate into mature bone cells. Manufacturers of existing osteoinductive implants tend to demineralise allograft material to expose BMPs within the bone to render the material osteoinductive.

The osteoinductive capacity of the bone implant as described herein is particularly surprising in view of the fact that the collagen-containing implant is treated to remove non-fibrous tissue proteins, such as BMPs, cytokines, chemokines and other growth factors. As such, it would be expected that any chemical molecular signals which could drive osteoinduction would be stripped from the bone implant during processing.

Indeed, Urist and Strates (J. Dent. Res. 1971 50: 1392-1406) noted that BMPs are inactivated by trypsin digestion. Harvesting and storage of the natural bone tissue material prior to processing would also be expected to have a detrimental effect on BMP and other growth factor activity. Buring and Urist (Clin. Orthop. Relat. Res. 1967 55: 225-34) further noted that gamma irradiation doses of 2 million to 4 million Roentogens (approximately 18 to 37 kGys) eliminates the potential for bone induction. Since both trypsin and gamma irradiation may be used in processing the bone implants described herein, it may be concluded that BMPs in the natural bone tissue material are significantly reduced (to sub-clinical levels) in the preparation of the bone implant materials according to the present invention, and that any remaining BMPs would be inactivated by the tissue processing.

Thus, it would be expected that exogenous factors such as BMPs would need to be added to the processed implant in order to restore osteoinductivity. Advantageously, however, the osteoinductive capacity of the bone implant as described herein does not rely upon the addition of exogenous osteoinductive factors such as growth factors. Thus, in some embodiments the bone implant may be free from exogenous osteoinductive factors.

It would seem that some signalling functionality remains despite the tissue processing. Although the reasons for these surprising observations are not entirely clear, and without wishing to be bound by any particular theory, it seems possible that host cells respond to ‘signals’ provided by the structure of the collagen (and/or small amounts of other fibrous tissue proteins of the bone implant) and/or mineral components, where present. It is possible that such signals may arise from a combination of different signalling elements provided by the collagen and/or small amounts of other fibrous tissue proteins and/or mineral components, where present.

This could result in recruitment of host cells and/or differentiation of host cells into osteogenic cells. The host cells could be, for example, stem cells, including mesenchymal stem cells and osteogenic stem cells, progenitor cells, such as osteoprogenitor cells, or any other host cells. The signals may be recognised directly by host cells. It is also possible that elements of the bone implant structure act indirectly on the host cells, perhaps by binding host growth factors or signalling molecules in a tissue-specific manner. The signals may reside in a combination of one or more primary, secondary, tertiary or quaternary structural elements of the fibrous tissue proteins of the implant and/or any mineral component. As such, signalling may be occurring through recognition of a combination of one or more of protein sequences, and one-dimensional topography, two-dimensional topography or three-dimensional topography. It is possible that different signalling elements of the fibrous tissue proteins and/or of any mineral component may cooperate to provide a signal.

The bone implant as described herein induces and guides the growth of bone tissue following implantation, providing for natural, ordered regeneration.

Thus, it is possible that the behaviour of host cells may be influenced and tissue growth guided by tissue-specific elements of the bone implant, in particular the collagen and/or other fibrous tissue proteins therein and/or any mineral component, giving rise to controlled, ordered bone regeneration.

The bone implant as herein described may also usefully be employed for in vitro growth and regeneration of bone tissue.

In particularly preferred embodiments, the bone implant materials described herein are remodellable such that controlled remodelling of the implant takes place following implantation into the host. Bone remodelling is essentially an interaction of two cellular activities: osteoclastic bone resorption and osteoblastic bone formation. The latter physiologic process not only maintains bone mass, skeletal integrity and skeletal function but is also the cellular process that determines structural and functional integration of bone substitutes.

The starting materials for the present invention may be obtained from any human or non-human mammal. In some embodiments, it is preferred that porcine bone tissue materials are processed to provide the bone implant, although it will be understood that other mammalian sources may alternatively be employed, such as primates, cows, sheep, horses and goats. Porcine cancellous bone is structurally similar to human bone, including with respect to trabecular bone architecture and remodelling activity (Mosekilde et al., 1987, Bone 14:379-382; Raab et al., 1991 J. Bone Miner. Res. 6:741-749; Thorwarth et al., 2005 J. Oral Maxilliofac. Surg. 63:1626-1633). Analysis of compact bone from different species has also shown that porcine and human bones have comparable Haversian systems in terms of diameter and area (Martiniakova et al., 2006 J. Forensic Sci. 51:1235-1239; Hillier and Bell, 1993 J. Forensic Sci. 52:376-382). Bone mineral content (BMC) and bone mineral density (BMD) of trabecular bone has been found to be slightly greater in porcine bone when compared to human bone (173 versus 76.3 mgs BMC and 373 versus 178 mg/cm³ BMD) (Aerssens et al, 1998, Endocrinology 139:663-670), whereas bone regeneration in pigs and humans appears similar, 1.2-1.5 mm per day and 1.0-1.5 mm per day respectively (Laiblin and Jaeschke, 1979, Berl Munch. Tierartztl. Wochenschr. 92:124-128).

Any suitable natural bone tissue material may be used as a starting material for production of a bone implant as described herein. Preferred bones for harvest include but are not limited to the femur, humerus and tibia or any other bone that provides an abundant source of cancellous or cortical bone.

The natural bone tissue material may comprise cancellous bone and/or cortical bone. Cancellous bone is generally ‘spongy’ with a relatively porous structure, which facilitates tissue processing and also allows for ready infiltration of the bone implant by host cells following implantation due to the porous interconnectivity of the bone matrix. This provides for good osteoconduction. Thus, cancellous bone may be the preferred starting material in some embodiments. However, cancellous bone tends to have relatively little inherent strength as compared to cortical bone. In contrast, cortical bone has a compact structure and is inherently strong. It may be therefore desirable to include bone implant material derived from cortical bone where the structural or mechanical performance of bone implant is of importance.

In some embodiments, the bone implant may be derived from natural bone tissue material which comprises a cancellous bone portion and a cortical bone portion. For instance, a block, wedge, or similar structure may be taken from a part of a bone comprising both cancellous and cortical tissue. It will be appreciated that the make-up of the bone implant may be varied depending upon the particular bone selected as a starting material and also the particular part of that bone selected for processing.

The density of the bone can be varied to alter the biomechanical and biological (healing) performance.

Whilst any appropriate processing methodology may be used, a particularly suitable process which may be adapted for use in preparing the bone implant is disclosed in U.S. Pat. No. 5,397,353, the contents of which are incorporated herein by reference. U.S. Pat. No. 5,397,353 describes processing of porcine dermal tissue to provide collagenous implant materials suitable for homo- or hetero-transplantation to repair soft tissue injuries. The implants retain the natural structure and original fibre architecture of the natural collagenous tissue from which they are derived, so that the molecular ultrastructure of the collagen is retained. The implant materials are non-reactive, any reactive pathological factors having been removed, and provide an essentially inert scaffold of dermal collagen.

It has now surprisingly been found that the processing techniques of U.S. Pat. No. 5,397,353 may be adapted for use in processing hard tissue, i.e. bone.

According to a further aspect of the present invention there is provided a process for the manufacture of a bone implant as herein described, which comprises treating natural bone tissue material to remove therefrom cells and cellular elements, non-fibrous tissue proteins, lipids and lipid residues, to provide a collagenous material displaying the original collagen fibre architecture and molecular ultrastructure of the natural bone tissue material from which it is derived.

As hereinbefore described, it is preferred that the processed bone implant retains at least part of the inorganic, mineral component of the starting material. In certain particularly preferred embodiments, at least a portion of the bone implant comprises mineral wherein the structure of the collagen-mineral composite of the starting material is at least partially maintained. The natural bone tissue material, or a part thereof, may be processed so as to preserve as much as possible of the structure of the collagen-mineral composite forming the bone. The substantial removal of non-fibrous tissue proteins, cells and cellular elements and lipids or lipid residues from the natural bone tissue material provides a composite of essentially collagen (with minor amounts of other fibrous tissue proteins) and mineral, in approximately the same arrangement as in the starting material.

Non-fibrous tissue proteins include glycoproteins, proteoglycans, globular proteins and the like. Cellular elements include antigenic proteins and enzymes and other cellular debris arising from the processing conditions. These portions of the natural tissue material may be removed by treatment with a proteolytic enzyme.

Whilst any proteolytic enzyme which under the conditions of the process will remove non-fibrous tissue proteins can be used, the preferred proteolytic enzyme is trypsin. It has previously been found that above 20° C. the treatment can in some circumstances result in an alteration of the collagen fibre structure leading to a lower physical strength. Moreover, low temperatures discourage the growth of microorganisms in the preparation. It is therefore preferred to carry out the treatment with trypsin at a temperature below 20° C. Moreover, trypsin is more stable below 20° C. and lower amounts of it may be required. Any suitable trypsin concentration may be used, for instance a concentration within the range of around 0.01 g/L to 25 g/L. It has been found that good results can be obtained using 2.5 g/L porcine trypsin, pH 8.

It will be appreciated that the reaction conditions for the treatment with trypsin may be routinely adjusted.

One method of removing lipids and lipid residues from the bone tissue is by the use of a selective enzyme such as lipase. A further, simpler and preferred method is solvent extraction using an organic solvent. Non-limiting examples of suitable solvents include non-aqueous solvents such as acetone, ethanol, ether, or mixtures thereof, acetone being preferred.

The process may be used to treat bone tissue material to provide a bone implant that is substantially free of non-fibrous tissue proteins, cellular elements, and lipids or lipid residues. Those substances said to be “substantially free” of materials generally contain less than 10% of, more typically less than 5% of, and preferably less than 1% of said materials.

A residual quantity of bone marrow lipids may remain in the processed bone implant, owing to the inherent difficulty in extracting these molecules from the centre of the bone. However, these lipids may act as a barrier to host cell infiltration of the bone implant, and so it is generally preferred that as much bone marrow lipid as possible be removed from the bone implant. Preferably, less than 10% of bone marrow lipids remain in the processed implant.

The bone tissue processing may optionally include a step of treatment with a cross-linking agent. Surprisingly, even in the presence of mineral component of the bone matrix, the collagen present in the bone tissue can be cross-linked. Cross-linking is known to reduce the immunogenicity of collagen.

Whilst any cross-linking agent may be used, preferred cross-linking agents include polyisocyanates, in particular diisocyanates which include aliphatic, aromatic and alicyclic diisocyanates as exemplified by 1,6-hexamethylene diisocyanate, toluene diisocyanate, 4,4′-diphenylmethane diisocyanate, and 4,4′-dicyclohexylmethane diisocyanate, respectively. A particularly preferred diisocyanate is hexamethylene diisocyanate (HMDI). Carbodiimide cross-linking agents may also be used, such as 1-ethyl-3-[3-dimethylaminopropyl]carbodiimide hydrochloride (EDC). Other possible cross-linking agents include glutaraldehyde, N-hydroxy succinimide (NHS), and hyaluronate polyaldehyde.

The extent of cross-linking may be adjusted by varying the concentration and/or duration of exposure to the cross-linking agent. Usefully, this may provide a mechanism for controlling the rate of bone remodelling following implantation.

By way of example, the bone implant may be cross-linked using HMDI. As a guide, the HMDI may be used at a concentration of around 0.01 g to 1 g per 50 g of approximate collagen weight in the tissue material. Typically, at least 0.1 g HMDI per 50 g of collagen is used. Cross-linking may be carried out over a range of different time periods. By way of example, the tissue may be exposed to the cross-linking agent for between around 1 hour and around 3 days. Typically, cross-linking is carried out for at least 12 hours, preferably at least 20 hours, such as around 24 to 72 hours.

It will be appreciated that the cross-linking conditions may routinely be varied in order to adjust the extent of cross-linking.

In one preferred embodiment of the present invention, the bone tissue is treated with a solvent, preferably acetone, a proteolytic enzyme, preferably trypsin, and optionally a cross-linking agent, preferably HMDI.

Preliminary data indicate that the mechanical properties may be altered depending on the level of cross-linking.

Typically, methods of bone processing described in the prior art involve the use of vacuums, high pressure or elevated temperatures to achieve the desired results (see, for example, U.S. Pat. No. 5,333,626, U.S. Pat. No. 5,513,662, U.S. Pat. No. 5,556,379, U.S. Pat. No. 5,380,826, and U.S. Pat. No. 5,725,579). In contrast, processing according to the present invention may successfully be carried out using essentially passive treatments in which no significant pressures or forces need be applied to the bone tissue. Treatment of the natural bone tissue material by the processing methods described herein with mild agitation results in a tissue material that is substantially free from cells.

The processed bone implant may be sterilised, for example by gamma-irradiation.

In preferred embodiments, the bone tissue is processed in a manner which substantially retains the mineral component of the natural bone tissue material. There is a risk that if the pH of the processing solutions is too low, the mineral component may dissolve and leech out of the bone implant. For this reason, the various processing steps may be carried out, for example, at an average pH of at least 7, such as about pH 8. Of course the pH may be further varied by routine experimentation.

The bone implant as described herein may take any suitable form. For instance, the bone tissue may be processed without making significant changes to the size or shape of the starting material. Thus, the bone implant as herein described may be provided as a structure approximating to the shape and dimensions of the bone used as the starting material. Alternatively, the size or shape of the bone tissue used as the starting material may be modified to provide different implants.

For example, in certain embodiments, the bone implant is provided as a bone piece or pieces of any desired size and shape. Bone pieces of any regular or irregular shapes may be provided, including, for example, chips, blocks, wedges, dowels and screws, or any other shapes envisaged by those skilled in the art. The bone tissue may be cut to size and/or shaped at any stage before, during, or after processing. Typically, the bone tissue material may be cut to the desired size and shape before any further processing is commenced, for instance using a saw or similar cutting instrument.

By way of example, it has been found that bone pieces of from about 5-50 mm³ to about 1 cm³ or larger are suitable for use as bone implants. The size may be routinely varied according to the nature of the application of the bone implant. It will be appreciated that the maximum size of any individual bone piece will be dictated by the size of the bone used as the starting material, although if necessary individual bone pieces may be joined together to provide larger implants.

According to a further aspect of the present invention there is provided a bone implant obtainable by a process as herein described.

According to a further aspect of the present invention there is provided a method of treatment comprising the step of surgically implanting into a patient a bone implant as herein described.

According to a further aspect of the present invention there is provided the use in bone surgery of a bone implant as herein described.

According to a further aspect of the present invention there is provided a bone implant as herein described for use in bone surgery.

According to a further aspect of the present invention there is provided the use of a bone implant as herein described for the manufacture of a product for use in bone surgery.

Embodiments of the present invention will now be described further in the following non-limiting examples with reference to the accompanying drawings, in which:

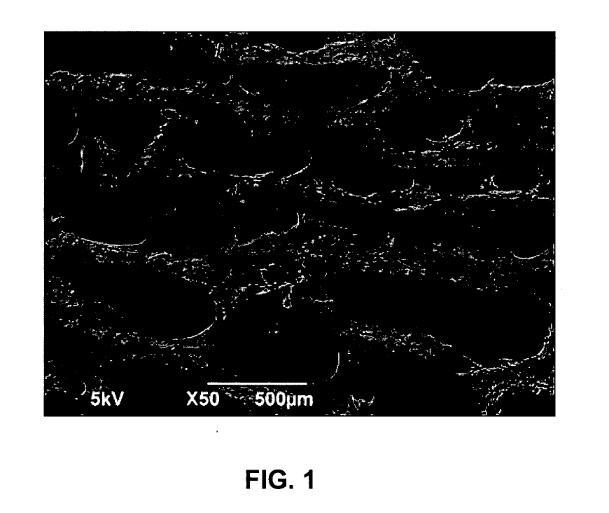

FIG. 1 is a scanning electron micrograph (×50 magnification) of a sample of a representative bone implant according to the present invention;

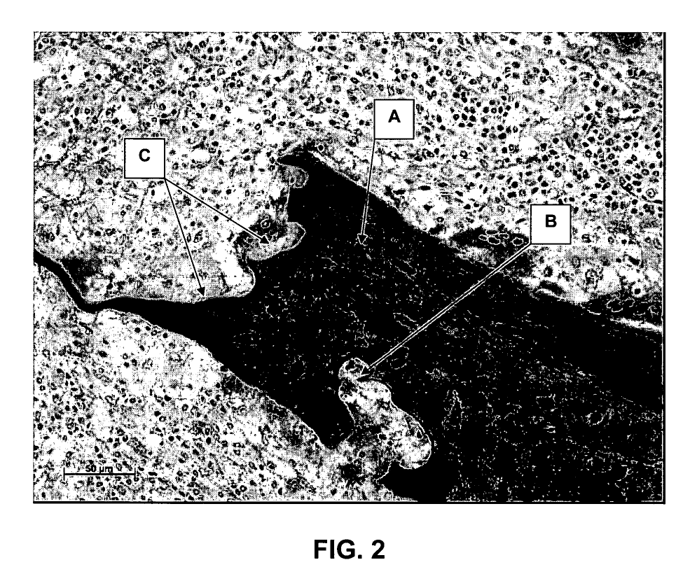

FIG. 2 is a photomicrograph (×200 magnification) of a representative bone implant according to the present invention 3 weeks post-implantation in a sheep critical size defect model, stained with toluidine blue and paragon;

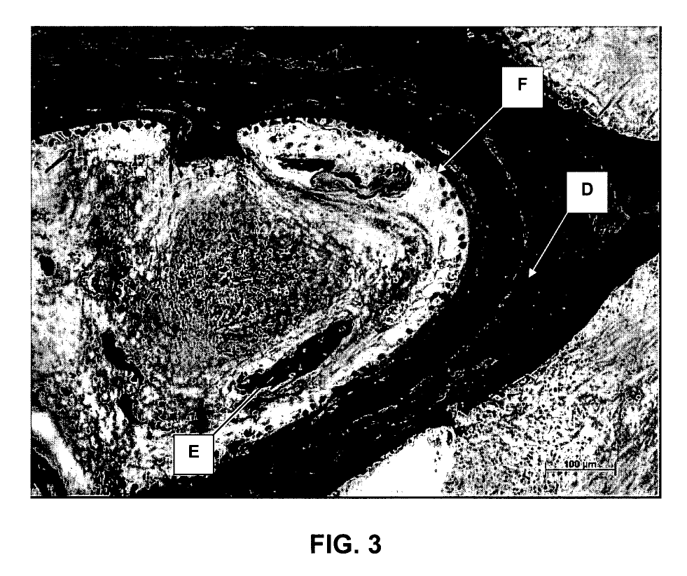

FIG. 3 is a photomicrograph (×200 magnification) of a representative bone implant of the present invention 3 weeks post-implantation in a sheep critical size defect model, stained with toluidine blue and paragon;

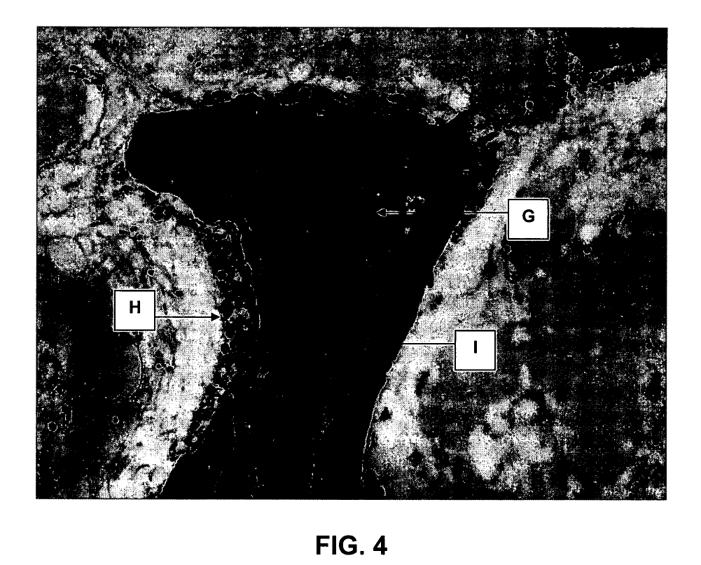

FIG. 4 is a photomicrograph (×200 magnification) of a representative bone implant according to the present invention 3 weeks post-implantation in a rabbit defect model, stained with toluidine blue and paragon;

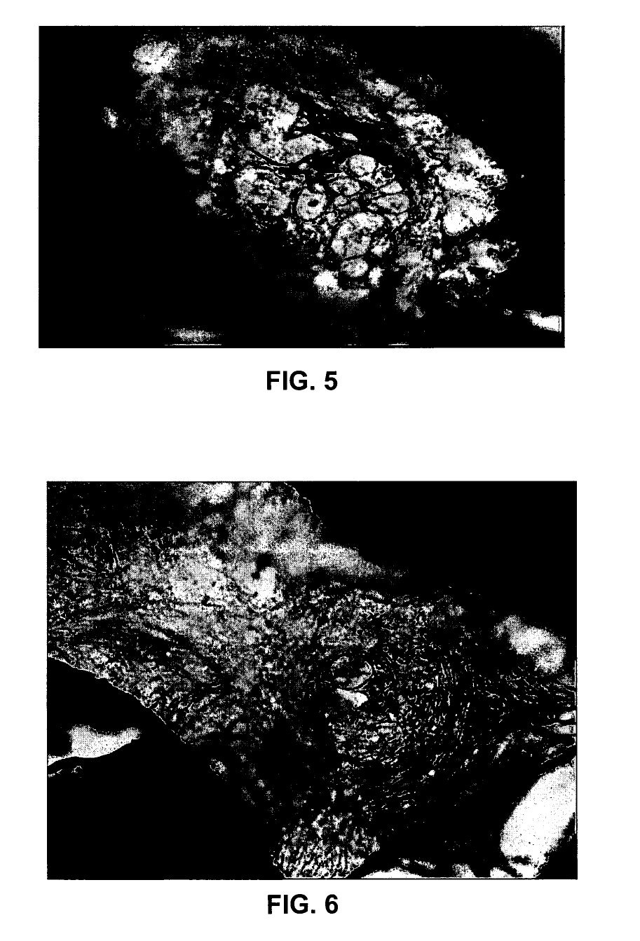

FIG. 5 is a photomicrograph (×400 magnification) of a section of a representative bone implant according to the present invention 6 weeks post-implantation intramuscularly in a rat, stained with haematoxylin and eosin;

FIG. 6 is a photomicrograph (×400 magnification) of a section of a representative bone implant according to the present invention 6 weeks post-implantation intramuscularly in a rat, stained with haematoxylin and eosin;

### EXAMPLES

### 1. Preparation of Bone Implant

Cancellous bone was harvested from the knee joint of a porcine hind limb. Harvesting was facilitated using a food grade band saw. All the cortical and cartilaginous material was cut from around the cancellous bone. The bone material was cut into pieces of around 1 cm³.

Upon completion of the harvesting process, the bone was then placed into acetone to remove lipids from the bone tissue. A 1-hour solvent rinse was followed by a 36-hour solvent rinse. The tissue was then rinsed thoroughly in 0.9% saline to remove the residual acetone from the structure. The material was then placed into trypsin at a concentration activity of 2.5 g/L, for a total duration of 28 days, after which the material was washed with saline to rinse away residual trypsin. After completion of the trypsin digestion, the bone was rinsed thoroughly in saline. The material was then washed in acetone. There followed a cross-linking step of treatment with HMDI in acetone. The amount of HMDI required was based on an approximation of the quantity of collagen present in the bone tissue, calculated on a weight basis assuming that 30% of the bone tissue is collagen. A concentration of 0.1 g HMDI per 50 g of collagen was added. The material was cross-linked for at least 20 hours, rinsed in acetone, and finally rinsed in saline. Samples were then gamma-irradiated at a minimum of 25 kGy.

For histological examination, samples were fixed in 10% neutral buffered formal saline. Following fixation, samples were processed, by routine automated procedures, to wax embedding. 10-micron resin sections were cut and stained with Giemsa. The sections of processed bone implant showed the retention of cancellous structure, retention of mineral and were totally devoid of any cellular presence. All of the natural septae, the lacuna and the canaliculi showed no presence of any cellular or tissue material and were seen as empty clear spaces.

For SEM analyses, samples of the bone implant were mounted onto SEM stubs using araldite glue. The samples were splutter coated with gold/palladium prior to examination at different magnifications. FIG. 1 shows the bone implant at a magnification of ×50. From this SEM image it can be seen that the bone implant has an open trabecular network with apparent pore interconnectivity and variable pore sizes. Trabecular thickness also varies and there is a high level of connected trabecular with approximately equal numbers of horizontal and vertical trabeculae.

### 2. Cross-Linking of Bone Implant

To quantify the effect of cross-linking on the resistance of the collagenous bone implant, a collagenase assay was used. This assay determines the level of resistance of a collagenous material to enzymatic digestion through weight difference.

By increasing the concentration of and exposure time to cross-linking agent, the collagenase resistance of the bone matrix was increased. This was not necessarily to be expected, since the mineral aspect of the bone would be expected to hinder access of cross-linking agent to collagen reactive sites.

### 3. Effect of Cross-Linking on Mechanical Properties of Bone Implant

Dowels of cancellous bone were manufactured to dimensions of around 8 mm×15 mm. The dowels were then treated with trypsin and acetone as in Example 1, to substantially remove the fats and non-collagenous proteins. Sixty dowels were separated into three groups of 20. Each group was then cross-linked to a different extent. Dowels of cross-linking Variant 1 were cross-linked using HMDI at a ratio of 0.1 ml HMDI per 50 g of collagen present, Variant 2 were cross-linked at 0.5 ml per 50 g of collagen present and Variant 3 were cross-linked at 1.0 ml of HMDI per 50 g of collagen present. In all cases cross-linking was carried out for approximately 20 hours.

The samples were then mechanically tested to determine the ultimate compression strength on an screw-driven Zwick Proline 500 test machine fitted with a 500N load cell with a an accuracy of 0.5 N.

Compression testing was completed using an environmental jig, which comprises a compression platten housed within a watertight bath. This allowed the samples to be tested in a physiological environment, i.e. while immersed in saline at 37° C. Load was applied axially to the samples at a crosshead speed of 0.1 mm/min.

Samples of a prior art bone implant (Orthoss® (Geistlich) were also tested by way of reference. Orthoss® is a commercially available bone implant derived from deproteinised bovine cancellous bone.

Surprisingly, the compression strength of the processed bone graft was altered with increasing levels of cross-linking agent. Furthermore, increased levels of cross-linking agent also altered the shear and fatigue characteristics of the bone implant.

In addition it was noted that the ultimate compression strength (UCS) was comparable to that of native human cancellous bone.

The UCS values for the processed bone graft were found to be within the central range of values reported for the UCS of fresh human cancellous bone, where values of between 0.5-13 MPa have been observed for the UCS. This compares favourably with the properties of allograft materials which can exhibit a 20-40% reduction in strength, as compared to fresh human bone, as a result of their processing and sterilisation procedures employed, particularly when freeze drying and gamma sterilisation are performed sequentially, as is common in bone banking. In contrast, the compressive strength of Orthoss® specimens fell toward the lower spectrum of data.

The performance of the Orthoss® implant suggests that the removal of the collagen from the bone tissue is detrimental to the mechanical performance of the implant. All cross-linked variants of the bone implant as described herein were found to have considerably greater compression strength than Orthoss®.

### 4. Analysis of BMP Content of Bone Implant

Samples of the bone implant of Example 1 were analysed for the presence of BMP-2. The samples were initially cryogenically milled to facilitate analysis of any BMPs present within the bone implant. Approximately 10 g of processed bone material were placed into an IKA analytical mill. Approximately 30-40 ml of liquid nitrogen was placed into the mill chamber with the bone material. The samples were left in the mill chamber with the nitrogen until cryogenically frozen. Once frozen, the bone was milled at a speed of approximately 20,000 rpm until finely ground. Any remaining nitrogen was allowed to evaporate to atmosphere, before the ground bone was transferred to a sterile universal container with a small volume of 0.9% saline.

For BMP-2 quantification analysis, the bone implant samples were digested with a collagenase solution overnight at a temperature of 37° C. Upon completion of the digestion, the samples were centrifuged and the protein supernatant was collected. An aliquot of the supernatant was then diluted for analysis by enzyme-linked immunosorbent assay (ELISA) (R&D Systems), following the manufacturer's standard instructions. A sample of rh-BMP-2 was used as a reference standard.

The results were compared with data available on three commercially demineralised bone matrices (DBM) (Wildemann et al. 2007 J Biomed Mater Res A, 81(2): 437-42). Wildemann et al. found that the commercially available DBMs contained on average 742 pg/μg (742 ppm) of bone morphogenic protein 2 (BMP-2). In contrast, the analysis completed on the bone implant of the present invention determined that it contained on average 0.05 ng/g (0.05 ppb) of BMP-2. This is significantly less than the commercially available products which are classed as osteoinductive. Thus, the bone implant according to the present invention can be considered to be substantially free from growth factors, and any BMPs present are in only trace amounts such that any activity level is essentially sub-clinical in performance.

### 5. Functional Implantation of Bone Implant

To investigate the healing and repair characteristics of the bone implant, a critical size defect (CSD) animal model was employed.

A CSD is an osseous defect which, if left untreated, shows less than 10% healing of bone during the lifetime of an animal. CSDs are therefore commonly used to provide models in which bone implants can be evaluated for their effectiveness in bone repair and healing.

The remodelling and healing characteristics of the bone implant of the present invention were compared to those of Orthoss®.

Twenty-one sheep were used for the study. These animals produced 25 defect sites at various time points, with each animal having up to four defects made in the medial femoral condyles. Sites were allocated to treatment groups using the bone implant of the present invention or Orthoss®, and empty defects, by random selection so that no animal had two test materials of the same type. Some sites were left ‘unused’. Five sample sites per group were investigated at each time point. Seven animals were allocated to each of three time points: 3 weeks, 6 weeks and 12 weeks.

Two holes were drilled, one in a proximal position and one in a distal position with more than 5 mm between the holes. The holes were drilled to a standard depth of 15 mm made with an 8.0 mm drill bit. Two 1 mm holes were drilled either side of the defect and 1 mm tantalum beads were inserted in order to correctly locate the defects on retrieval using radiography. After irrigating with sterile saline, the appropriate test material was pressed into place, or for the empty sample group and unused sites the defects were left empty. The wound was closed and the contra-lateral medial femoral condyle exposed by a medial approach. In a similar manner two holes were drilled, irrigated with sterile saline, test materials inserted and the wound closed.

At the allotted time point, the animals were humanely euthanised and the entire implant including at least 5 mm of surrounding bone was removed from the femur. The samples were defatted prior to being embedded in resin, sliced and analysed histologically using toluidine blue and paragon staining. Fluorescent bone markers previously injected into the animals were used to quantify bone remodelling adjacent to the defects and within the implant materials. The uptake of markers at sites of bone mineral deposition provided a means of demonstrating regions of active bone formation and mineralisation. In all groups, peripheral measurements of bone turnover rates were calculated from two random regions along one side of the defect and two areas from the opposite side (four in total). Four other random regions were selected within each of the defects and measurements. Turnover rates were calculated in μm day⁻¹.

The results showed that more new bone was measured within the defects repaired with samples of the bone implant of the present invention relative to the Orthoss® samples. At the 12-week time point significantly more new bone was measured in the bone implant samples (35.968%) when compared with the Orthoss® samples (19.588%). In addition, the bone graft resorbed in a controlled manner as new bone was formed.

Resorption of the bone implant is important to prevent alteration to the material properties of the bone within the graft site once the healing process is completed. FIG. 2 shows that the bone graft (A) had ‘scalloped’ areas (B) after 3 weeks' implantation in a critical sized defect in an ovine model. This ‘scalloping’ is typical in normal bone remodelling through the action of osteoclasts (C).

With the Orthoss® material, after 6 weeks' implantation it was apparent that although new bone was laid down, there was no evidence of scalloping and, therefore, osteoclastic activity was not evident showing the implant was bioinert.

With the bone implant of the present invention, there was a change in the appearance of the bone implant at the 12-week time point compared to the three-week time-point. The density of the bone implant material was reduced and the topography started to resemble that of the host cancellous bone structure.

FIG. 3 shows intramembranous bone formation in the soft tissue adjacent to the bone implant (D). In these regions (E) bone had not formed directly on the implant surface but instead had formed on collagen fibres through intramembranous ossification. This suggests a possible osteoinductive component within the environment. Osteoblasts actively laid down osteoid (F).

In addition to the critical size sheep defect study, smaller bone dowels were also prepared (4 mm diameter) and processed in accordance with the present invention. They were implanted into the condyles of the right and left knee of adult (greater than 2 kg) female New Zealand white rabbits. These implants were inserted by making an incision lateral to the patella over the femoral condyles, measuring 3 cm. The patella was reflected medially exposing the trochlear groove of the knee joint. A pilot hole measuring 2 mm was drilled to a depth of around 6 mm through the trochlear groove of the knee joint. The bone dowels were inserted and press-fitted into place. The patella was repositioned and the wound closed with resorbable Vicryl® in two layers. The procedure was repeated on the other knee joint. The animals were sacrificed after 21 days and their femoral condyles prepared for histology using toluidine blue and paragon staining.

Histology data from this study further exemplifies natural bone turnover with the bone implant according to the present invention. FIG. 4 shows the presence of osteoblast seams (H) ‘scalloping’ the implant (G) along with new bone (I) laid down onto the surface of the bone implant. These cellular activities are demonstrative of a natural biological response.

### 6. Intramuscular Implantation of Bone Implant

Pieces of the decellularised collagen-containing bone implant of Example 1 were implanted intramuscularly into rats. For implantation, slices of approximately 0.2 cm were cut from the 1 cm³ pieces of bone implant.

Male Wistar rats were pre-medicated according to species and weight. General anaesthesia was induced and maintained using agents appropriate for species and size. Sterile technique was used. A dorsal cranio-caudal skin incision was made just lateral to the spine from a point 1 cm distal to the edge of the scapula extending approximately 1.5 cm distally. The psoas muscle was identified, exposed and divided longitudinally on each side to provide 2 intramuscular ‘pockets’. Haemostasis was maintained by careful dissection; no electrocautery was used. Samples of processed bone (approximately 1 cm×1 cm×0.2 cm) were implanted into each of the psoas muscle pockets. The psoas muscle pockets were closed with Vicryl® sutures and to complete the procedure the dorsal midline incision was then closed with interrupted sutures.

Six weeks after surgery, the bone implant was explanted together with the surrounding tissue and immediately fixed in 10% neutral buffered formal saline. Following fixation, samples were processed, by routine automated procedures, to wax embedding. 5-micron or 10-micron resin sections were cut and stained with Giemsa and/or haematoxylin and eosin.

The bone implant was observed to be well integrated into the tissue, with no signs of an elevated immune response. There was a narrow band of mainly fibroblastic inflammatory response immediately adjacent to the bone implant which occasionally extended a small distance into the muscle. Within this response there were some polymorphs, macrophages and the occasional monocyte. These features represent a normal ‘foreign body’ tissue response as would be seen with any non-immunogenic implant even an autograft. The implanted bone implant retained its structure with easily definable morphological features, including calcified cancellous component and well preserved lacunae. The overall integrity of the implant was also well preserved.

Within most of the lacunae, the septae and the cannaliculi of the implanted bone implant samples there were thin, fibrinous, stranded structures within which there were a variety of cells including fibroblasts, polymorphs, monocytes and some larger mononuclear cells of indistinct lineage. In some of the lacunae there were large, mononuclear cells with recognisable nucleoli, which showed features of early osteocytic lineage (see FIGS. 5 and 6). This was a surprising result, given that the tissue processing ostensibly renders the bone implant inert, removing non-fibrous tissue proteins, such as growth factors. It would seem that the bone implant retained some signalling functionality. It was particularly surprising that this was apparently sufficient to influence the recruitment and/or development of osteocytic host cells in an intramuscular environment. Cells of this type would not be expected to be present at the host implant site. It is possible that the host cells were derived from progenitor cells, perhaps from the fibroblast milieu, although the exact mechanisms involved are unclear. The bone implant may retain tissue-specific signals in elements of fibrous tissue protein sequence or conformation, which signals are able to influence host cell behaviour within the bone implant, either directly or indirectly.

By way of further example an additional intramuscular study was completed comparing the bone implant of Example 1 with Orthoss® and a demineralised version of the bone implant of Example 1. Each of the materials for evaluation was trimmed to approximately 1 cm×1 cm×0.5 cm. These samples were separately implanted into intramuscular pockets on the latero-ventral aspect of rats. Samples were explanted at 2 months and at 3 months. Samples were explanted together with the adjacent surrounding tissues and fixed in 10% neutral buffered formal saline. Once fixed, the entire sample was de-calcified, a block from the centre of the explant, to include the implant and all surrounding tissue, was processed to paraffin wax embedding by routine automated procedures. Two 5-micron sections were cut from each block, one was stained with haematoxylin and eosin and one with picrosirius red together with Millers elastin stain. Sections were examined using a transmitted light microscope with polarizing ability.

Both the demineralised bone implant and Orthoss® elicited an immune reaction, with host cells breaking down the implanted devices.

The bone implant of the present invention did not cause a foreign body inflammatory response and evidence of neo-collagenesis in the inter-trabecular spaces was identified. This may indicate early osteogenesis.

It is of course to be understood that the invention is not intended to be restricted by the details of the above specific embodiments, which are provided by way of example only.

1. A bone implant derived from natural bone tissue material, wherein the bone implant is substantially free of non-fibrous tissue proteins, cells and cellular elements and lipids or lipid residues and comprises collagen displaying original collagen fiber architecture and molecular ultrastructure of the natural bone tissue material from which it is derived.

2. A bone implant according to claim 1, wherein at least a portion of the bone implant comprises bone mineral derived from the natural bone tissue.

3. A bone implant according to claim 2, wherein the bone implant comprises approximately 20 to 75% organic material.

4. A bone implant according to claim 3, wherein the bone implant comprises approximately 22 to 50% organic material.

5. A bone implant according to claim 4, wherein the bone implant comprises approximately 25 to 35% organic material.

6. A bone implant according to claim 2, wherein the bone implant is substantially non-demineralised.

7. A bone implant according to claim 2, wherein the bone mineral displays original mineral architecture of the natural bone tissue material.

8. A bone implant according to claim 7, wherein the collagen and bone mineral have a structural relationship approximating to the natural bone tissue material.

9. A bone implant according to claim 1, wherein the bone implant structure is an open network of connected bone trabeculae with interconnected pores.

10. A bone implant according to claim 1, wherein the bone implant has a porosity of between around 5 to 90%.

11. A bone implant according to claim 1, wherein the bone implant has pores of between 1 μm and 2000 μm.

12. A bone implant according to claim 11, wherein the bone implant has pores of between 100 μm and 1000 μm.

13. A substantially non-demineralised bone implant derived from natural bone tissue material, wherein the bone implant is osteoconductive and osteoinductive.

14. A bone implant according to claim 1, wherein the bone implant is remodellable.

15. A bone implant according to claim 1, wherein the natural bone tissue material is porcine bone tissue.

16. A bone implant according to claim 1, wherein the natural bone tissue material comprises cancellous and/or cortical bone.

17. A process for the manufacture of a bone implant which comprises treating natural bone tissue material to remove therefrom cells and cellular elements, non-fibrous tissue proteins, lipids and lipid residues, to provide a collagenous material displaying the original collagen fiber architecture and molecular ultrastructure of the natural bone tissue material from which it is derived.

18. A process according to claim 17, wherein the process comprises a step of treatment with a proteolytic enzyme.

19. A process according to claim 18, wherein the proteolytic enzyme is trypsin.

20. A process according to claim 17, wherein the process comprises a step of removing lipids and lipid residues by solvent extraction using an organic solvent.

21. A process according to claim 20, wherein the solvent is selected from acetone, ethanol, ether, or mixtures thereof.

22. A process according to claim 17, wherein the process comprises a step of treatment with a cross-linking agent.

23. A bone implant obtainable by a process according to claim 17.

24. A method of treatment comprising the step of surgically implanting into a patient a bone implant according to claim 1.

25. Use in bone surgery of a bone implant according to claim 16.

26. A bone implant according to claim 23 for use in bone surgery.

28. Use of a bone implant according to claim 23 for the manufacture of a product for use in bone surgery.
